(19) United States
(12) Patent Application Publication
(10) Pub. No.: US 2024/0093837 A1
(43) Pub. Date: Mar. 21, 2024

(54) Gas Blending Apparatus and Gas Delivery System with such a Gas Blending Apparatus

(71) Applicant: OXFO Corporation, Chestnut Hill, MA (US)

(72) Inventors: Carlos Fernando Bazoberry, Chestnut Hill, MA (US); Brent H. Young, Boston, MA (US)

(73) Assignee: OXFO Corporation, Chestnut Hill, MA (US)

(21) Appl. No.: 18/371,127

(22) Filed: Sep. 21, 2023

Related U.S. Application Data
(60) Provisional application No. 63/408,655, filed on Sep. 21, 2022.

(51) Int. Cl. F17C 13/04; F17D 3/05

(52) U.S. Cl. CPC F17C 13/04; F17D 3/05; F17D 1/04

(57) ABSTRACT

A gas blending apparatus and an ambient pressure gas delivery and conservation system with such a gas blending apparatus for delivering gas at ambient pressure from a donor reservoir to a recipient. A main conduit body has a first port in fluidic communication with the donor reservoir and a second port for supplying gas to the recipient through ambient pressure tubing. A one-way inspiratory valve between the first port and the second port permits gas to be drawn from the reservoir but not to be received through the second port, through the first port, and into the donor reservoir. An air-input orifice permits ambient air to be blended with gas drawn from the reservoir. An orifice adjustment member is selectively repositionable relative to the air-input orifice to adjust the effective size of the aperture thereby to adjust the ratio of ambient air to gas drawn from the reservoir.

### RELATED APPLICATION

This application claims priority to U.S. Provisional Patent Application No. 63/408,655, filed Sep. 21, 2022, which is incorporated herein by reference.

### FIELD OF THE INVENTION

The present invention relates generally to the delivery of gases from a source to a recipient. More particularly, disclosed herein is a gas blending apparatus for use within a system for delivering oxygen or another gas or mixture of gases to a recipient, the gas blending apparatus operative to permit direct and immediate control over the fraction of inspired oxygen (FiO2) or other gas or gases delivered to the recipient.

### BACKGROUND OF THE INVENTION

Normally, the lungs absorb oxygen in sufficient supply from the air during natural breathing. However, certain conditions can prevent a person from getting enough oxygen. As a result, oxygen therapy with oxygen delivery equipment is required. Patients can receive oxygen therapy from a source of oxygen through tubes resting in their nose, through a facemask, or through a tube placed in their trachea or windpipe. Oxygen treatment increases the amount of oxygen the lungs receive and deliver to the blood. Oxygen therapy may be prescribed for a patient when the patient has a condition that causes the patient's blood oxygen levels to be too low. Low blood oxygen may make patients feel short of breath, tired, or confused and can damage the patient's body. Oxygen therapy may be needed on a temporary basis, such as due to a treatable respiratory illness, or on a long-term basis. Often, the source of oxygen is a tank of compressed oxygen gas or liquid.

The supply of oxygen can be a critical need for hospital patients and others. Meanwhile, in developing countries and during times of increased demand in all places, shortages of oxygen and excessive costs can place extreme limits on availability and can jeopardize the health and safety of patients in need. For instance, during the COVID-19 pandemic, the demand for oxygen left hospitals and other caregiving institutions in dire need of the life-saving gas. One headline from the AP News network on Jun. 24, 2020 warned, “Scarce Medical Oxygen Worldwide Leaves Many Gasping for Life.” One day later, Reuters observed, “WHO Warns of Oxygen Shortage as COVID Cases Set to Top 10 Mln” with the World Health Organization estimating based on there being approximately one million new coronavirus cases worldwide per week that the world will need 620,000 cubic meters of oxygen per day, which roughly equaled 88,000 large cylinders, for COVID-19 patients alone.

One way that supplemental oxygen is supplied to patients under the teachings of the prior art is via a fluidic connection, typically tubing, between a pressurized source of oxygen, such as an oxygen cylinder or tank, and an output interface, such as a nasal cannula or mask, to the patient to provide high flows of oxygen. In such systems, the oxygen flows continuously, regardless of whether the patient is breathing in or out. As a result, even while the patient exhales and cannot intake oxygen, the oxygen flows constantly. Huge volumes of oxygen are thus wasted. Indeed, half or even more of the constantly supplied oxygen is wasted and is simply expelled to the atmosphere. During exhalation, the entirety of the supplied oxygen is wasted, and a portion of the supplied oxygen is often wasted even during inhalation.

High-flow systems inherently provide an excess supply of oxygen to ensure that the patient has sufficient oxygen over the entire respiratory cycle. Concomitantly, it will be recognized that the ability to conserve oxygen with one patient may well save the life of another, particularly in exigent circumstances, such as during an epidemic or a pandemic involving respiratory distress, where need can dangerously outpace supply. In remote and economically challenged locations, replenishing oxygen supplies can be highly costly or even catastrophically impossible. The challenge of providing oxygen in sufficient supply while minimizing waste is well recognized.

In a typical nasal cannula configuration, one end of an oxygen supply tube is connected to the source of oxygen while the other end of the tubing splits into two branches that meet to form a loop. Two nasal prongs are positioned along the loop for insertion into a patient's nares. Oxygen continuously flows through the tubing to exit through the nasal prongs and into the patient's nares. During inspiration, the patient thus inhales oxygen through the prongs together with entrained room air that is drawn through the space between the nasal prongs and the walls of the patient's nares. During exhalation, the patient exhales through the space between the nasal prongs and the walls of the patient's nares while oxygen continues to exit into the patient's nares. Much of that oxygen is carried with the expiratory flow into the surrounding room air.

In continuous flow systems, the fraction of inspired oxygen (FiO2) provided to the patient is sought to be controlled by increasing or decreasing the oxygen flow through the oxygen supply tube. Disadvantageously, once the flow rate is set, it works optimally only for the breathing pattern of the patient at the time of calibration. Changes to that breathing pattern, such as through physical exertion or another change in circumstance, will affect the FiO2, which will in turn affect the saturation of the patient. For example, when a patient takes a deeper breathe or is exerting and breathing more frequently as a result of exertion, the patient will inhale a larger volume into his or her lungs. Since the volume of oxygen provided is fixed but the volume of air is not, more air enters the blend, more dilution occurs, and FiO2 is reduced. The patient's oxygen-blood saturation (SaO2) may thus decrease with decreased FiO2. Active readjustment from the patient or medical personnel may be required. However, patients may not possess the skills or knowledge necessary to make such adjustments accurately, or they may simply be unaware or inattentive to the need to do so. Moreover, medical personnel may be unavailable to make continual adjustments to oxygen flow, particularly in non-medical settings.

In attempting to confront the foregoing, pulsed oxygen delivery systems have been disclosed to attempt to conserve oxygen by sensing the patient's breathing cycle and delivering a short-duration flow or pulse of oxygen during inhalation. However, such systems rely on complex circuitry and operation and may not adequately approximate natural human breathing. In pulsed oxygen delivery, oxygen is “pulsed” to the patient in one bolus of oxygen during the inhalation phase.

Multiple important factors come into play in supplying consistent and effective supplemental oxygen to patients. By way of example and not limitation. FiO2 is affected by oxygen purity, the trigger mechanism, the pulsed dose, the duration of the pulse, the pulsed flow curve, the ventilation rate, and inspiratory peak flow. No known device has optimized all of these factors perfectly. Instead, each manufacturer is forced to make trade-offs among them.

Pulsed oxygen delivery systems are typically set to trigger the pulse dose upon a specific but non-standardized negative pressure in the inspiratory curve of the breathing cycle. If the triggering pressure is too low, then the pulse dose will be delivered too early in the inspiratory curve when a patient does not have enough negative pressure to inhale the full amount of oxygen delivered. If the triggering pressure is too high, then the pulse is delivered too late in the inspiratory curve for optimal clinical use. Highly complex engineering, hardware, and software would be required for pulse oxygen delivery systems to respond based on real-time biofeedback from the patent to respond dynamically and with greater sensitivity to negative pressure and the patient's respiratory cycle and volume. To be fully responsive, the system would need to respond immediately or nearly immediately in what can be referred to as a “shadow effect” to meet the fluctuating needs of daily life, such as when a person transitions from sitting, to standing, to walking, to talking, to exercising, and other daily tasks. Current systems do not do this satisfactorily, and it will again be noted that current supplemental oxygen systems typically require the patient to remember to adjust the rate of flow and to do so based on what essentially amounts to a best guess based on changing activity levels or respiratory rates. These and further factors render patients using pulsed oxygen systems vulnerable to desaturation and resulting discomfort and health risks.

With a knowledge of the concomitant yet competing needs for conserving oxygen while being able to provide ample volumes of the same on demand, the present inventors developed the Automatic System for the Conservation of Gas and other Substances of application Ser. No. 17/068,718, filed Oct. 12, 2020, which is incorporated herein by reference. The automatic conservation system is operative to provide ample oxygen on demand to patients while conserving against loss and waste, including during the expiratory breathing phase. The automatic conservation system minimizes the oxygen consumption of individual patients while meeting patient needs and maximizing the effective supply of oxygen. In so doing, the automatic conservation system enables better health outcomes in a cost-efficient manner, even in times of public health crises.

In practices of the system, an expandable and compressible donor reservoir retains a volume of oxygen at ambient pressure. A supply conduit receives oxygen from a source of oxygen, and an ambient pressure conduit supplies oxygen from the donor reservoir to a patient through an ambient pressure conduit. An inflation detection system detects when the donor reservoir is inflated with oxygen to a predetermined state of inflation, such as within a range of a fully inflated condition, and when the donor reservoir is below the predetermined state of inflation. When the donor reservoir is inflated to the predetermined state of inflation, the valve system is closed to prevent oxygen from flowing from the source of oxygen and into the donor reservoir. When the donor reservoir is below the predetermined state of inflation, the valve system is opened to permit oxygen to flow from the source of oxygen to replenish the donor reservoir automatically. Oxygen can thus be continuously retained in the reservoir and supplied on demand to a patient through a patient interface delivery device, such as a nasal cannula or a breathing mask, with minimized waste. By providing such an on-demand oxygen supply system in place of, for instance, a high-flow oxygenation system, massive reductions in oxygen requirements and waste are realized.

However, in view of the their development of systems for supplying oxygen and other gases at ambient pressure in an on-demand, the inventors have appreciated further needs and opportunities in the supply of gases in that format. Among the most significant of those is the ability to exercise direct and immediate control over the ratio of oxygen and entrained air provided during inspiration. While doing so could additionally or alternatively be carried out at the nasal cannula or breathing mask, the present inventors have appreciated that advantage may be had by doing so proximal to or within the apparatus from which the oxygen or other gas is delivered. The inventors have further appreciated that there is a need to ensure that expiratory breath is prevented from being returned into the delivery apparatus and particularly into the donor reservoir to mix with the oxygen or other gas retained therein.

### SUMMARY OF THE INVENTION

With a recognition of the foregoing, the present inventors set forth with the basic object of providing a gas blending apparatus particularly adapted for use with an ambient-pressure gas delivery and conservation system.

A more particular object of embodiments of the invention is to provide a gas blending apparatus for an ambient-pressure gas delivery and conservation system that permits direct and immediate control over the ratio of oxygen and entrained air provided during inspiration.

A further object of embodiments of the invention is to provide a gas blending apparatus for an ambient-pressure gas delivery and conservation system that permits control over the ratio of oxygen and entrained air provided during inspiration proximal to or within the apparatus from which the gas is delivered.

Another particular object of embodiments of the invention is to provide a gas blending apparatus for an ambient-pressure oxygen delivery and conservation system operative to prevent exhausted breath from being returned into the oxygen delivery apparatus or into to a donor reservoir retained therewithin.

Yet another object of the invention in particular embodiments is to provide a gas blending apparatus and an ambient-pressure oxygen delivery and conservation system using such a gas blending apparatus that enables the achievement of a “shadow effect” wherein oxygen or another gas is automatically provided at a desired saturation on demand with every breath without regard to respiratory frequency, volume, or other factors to adjust to a patient's breathing pattern immediately and automatically.

A further object of the embodiments of the invention is to provide a gas blending apparatus and an ambient-pressure oxygen delivery and conservation system using such a gas blending apparatus that enable a consistent fraction of inspired oxygen (FiO2) or other gas or gases to be provided without the need for complex mechanical or software systems.

An additional object of the embodiments of the invention is to provide a gas blending apparatus and an ambient-pressure oxygen delivery and conservation system using such a gas blending apparatus that permit a patient to maintain a desired oxygen-blood saturation (SaO2) including during changes in respiratory frequency and volume.

These and further objects and advantages of the present invention will become obvious not only to one who reviews the present specification and drawings but also to those who have an opportunity to experience the gas blending apparatus and the ambient pressure oxygen delivery and conservation system using such a gas blending apparatus in operation. However, it will be appreciated that, while the accomplishment of plural of the foregoing objects in a single embodiment of the invention may be possible and indeed preferred, not all embodiments will seek or need to accomplish each and every potential advantage and function. Nonetheless, all such embodiments should be considered within the scope of the present invention.

In carrying forth one or more of the foregoing objects, one embodiment of the gas blending apparatus is adapted for use with an ambient pressure gas delivery and conservation system, which may alternatively be referred to as an ambient pressure gas dispensing and conservation system, with ambient pressure tubing for providing gas at ambient pressure from a donor reservoir to a recipient. The gas blending apparatus comprises a main conduit body with an inner volume, a first port for being disposed in fluidic communication with the donor reservoir, and a second port for supplying gas to the recipient. A one-way inspiratory valve is disposed within the main conduit body between the first port and the second port. The one-way inspiratory valve is operative to permit gas to be drawn in from the donor reservoir through the first port and passed through the second port to the recipient. The one-way inspiratory valve is also operative to prevent gas from being received through the second port and passed through the first port into the donor reservoir. For avoidance of doubt, the foregoing is intended to be in the conjunctive, meaning that the one-way inspiratory valve operates to prevent gas, such as exhaled breath, from flowing through the main conduit body and into the donor reservoir. An air-input orifice is disposed in the main conduit body distal to the one-way inspiratory valve with respect to the first port. The air-input orifice is operative to provide an aperture into the inner volume of the main conduit body with an effective size, and this aperture permits the entrance of ambient air into inner volume of the gas blending apparatus, such as on inhalation by the recipient. Ambient air can thus be drawn into the main conduit body through the air-input orifice and blended with gas drawn from the donor reservoir in a ratio of ambient air to gas drawn from the donor reservoir.

In embodiments of the gas blending apparatus, a neck connector is engaged with the main conduit body for coupling the first port of the main conduit body with the donor reservoir. For instance, the neck connector can be matingly engaged with the main conduit body and sealingly received into a neck formed in the donor reservoir.

Also as disclosed herein, a one-way valve is fitted to the air-input orifice. The one-way valve fitted to the air-input orifice is operative to permit ambient air to be drawn into the inner volume of the main conduit body but to prevent gas from being exhausted through the air-input orifice.

According to embodiments of the gas blending apparatus, the effective size of the aperture provided by the air-input orifice is selectively adjustable. By adjusting the effective size of the aperture provided by the air-input orifice, an adjustment of the ratio of ambient air drawn through the air-input orifice to gas drawn from the donor reservoir through the first port can be achieved. Again for avoidance of doubt, reference to the effective size of the aperture provided by the air-input orifice is intended to refer to the effective total size of the opening passage through the air-input orifice. This includes, but is not necessarily limited to, adjusting the size of the orifice itself or adjusting the size of the opening passage provided through the orifice. The effective size of the aperture provided by the air-input orifice can be selectively adjustable manually, or the effective size of the aperture could be adjusted automatically.

In one practice of the invention, the effective size of the aperture provided by the air-input orifice can be selectively adjustable manually by a selective repositioning of an orifice adjustment member that is selectively engaged with the main conduit body to overlap with the air-input orifice. The orifice adjustment member is repositionable in relation to the main conduit body to adjust the effective size of the aperture into the inner volume of the main conduit body provided by the air-input orifice. The effective size of the aperture into the inner volume of the main conduit body could be adjusted in any manner, including but not limited to by plural different apertures in the orifice adjustment member, by one or more continuous apertures of varying dimension so that portions thereof can be aligned with the air-input orifice, by an orifice adjustment member with a portion thereof that is adjustable in overlap with the air-input orifice, or in another manner as would be obvious to one of ordinary skill in the art after reading this disclosure. In any such construction, adjustment of the effective size of the aperture into the inner volume of the main conduit body enables direct and immediate control over the ratio of ambient air to gas drawn through the first port from the donor reservoir.

For instance, the orifice adjustment member can have a plurality of apertures in spaced relation to one another for being selectively aligned with the air-input orifice to adjust the effective size of the aperture provided by the air-input orifice. The orifice adjustment member has a plurality of aperture locations in spaced relation to one another that establish different effective opening sizes for being selectively aligned with the air-input orifice. Within the scope of the foregoing, the aperture locations can each have a single, differently-sized aperture, or the aperture locations could have differing numbers of similarly or differently sized or shaped apertures that in cumulation provide a different total open area.

In certain embodiments, the orifice adjustment member comprises a cylindrical member that is selectively repositionable in relation to the main conduit body to overlap with the air-input orifice. The cylindrical member has a plurality of differently sized apertures in spaced relation for being selectively aligned with the air-input orifice to adjust the effective size of the aperture provided by the air-input orifice and thereby to provide direct and immediate control over the ratio of air drawn through the air-input orifice in relation to gas drawn from the donor reservoir. For instance, differently sized apertures in the cylindrical member can be circumferentially spaced around the cylindrical member, longitudinally spaced along the cylindrical member, or otherwise disposed. While other configurations are possible, the differently sized apertures spaced around the cylindrical member can vary in size sequentially.

In practices of the invention, the cylindrical member of the orifice adjustment member comprises a portion of an output connector matingly engaged with the main conduit body. In such embodiments, the output connector can further comprise a tubular portion that extends beyond the main conduit body for connecting to an ambient pressure conduit for providing gas to the recipient.

To permit the cylindrical member to be disposed and retained in a known position relative to the main conduit body, mechanical engagement formations can be retained on at least one of the cylindrical member and the main conduit body. Furthermore, visual setting indicators can be retained to move with the cylindrical member and at least one visual setting indicator can be retained on the main conduit body for permitting the effective size of the aperture provided by the air-input orifice to be adjusted in a known manner.

In embodiments of the gas blending apparatus, an injection port is disposed in the main conduit body. The injection port can be connected to a supply source, such as a source of compressed oxygen, through high-pressure tubing. The injection port is disposed proximal to the one-way inspiratory valve with respect to the first port whereby gas received from the supply source is directed into the donor reservoir to replenish and inflate the donor reservoir.

Manifestations of the invention can be alternatively characterized as a gas delivery system for providing gas to an individual. The gas delivery system, which can be an ambient pressure gas delivery or dispensing system, has a donor reservoir that is adapted to retain gas. The donor reservoir has a fully inflated condition. A supply valve is disposed in fluidic association with the donor reservoir. The supply valve has an open condition wherein gas is allowed to flow into the donor reservoir and a closed condition wherein gas is not allowed to flow into the donor reservoir. An inflation detection system is operative to detect when the donor reservoir is inflated to within a predetermined range of the fully inflated condition. The inflation detection system detects a first condition when the donor reservoir is inflated to within the predetermined range of the fully inflated condition, such as at or within some range below the fully inflated condition. The inflation detection system also detects a second condition when the donor reservoir is inflated below the predetermined range of the fully inflated condition, and the inflation detection system is operative to actuate the supply valve to the open condition when the donor reservoir is inflated below the predetermined range of the fully inflated condition to cause an inflation of the donor reservoir. A gas blending apparatus as previously described has an air-input orifice that is operative to provide an aperture into the inner volume of the main conduit body with an effective size to permit the entrance of ambient air into inner volume of the gas blending apparatus. Under such constructions, ambient air can be drawn into the main conduit body through the air-input orifice and blended with gas drawn from the donor reservoir in a ratio of ambient air to gas drawn from the donor reservoir.

In practices of the invention disclosed herein, the donor reservoir has an outer wall, an inner volume for retaining a volume of oxygen or other gas, and at least one orifice for allowing a passage of gas into and out of the inner volume. The donor reservoir can, for example, comprise a shell of flexible material, such as a shell of foil. A supply conduit is adapted to receive oxygen from a source of oxygen. The supply conduit has a first end for supplying oxygen to the donor reservoir and a second end for being fluidically connected to the source of oxygen, and an ambient pressure conduit is adapted to supply oxygen along a fluid path from the donor reservoir to a recipient, such as through a nasal cannula or a breathing mask. The ambient pressure conduit has a first end in fluidic communication with the donor reservoir, such as through a connector, for receiving oxygen from the donor reservoir and a second end for being fluidically connected to the recipient.

In certain embodiments, the inflation detection system comprises an electro-mechanical system. For instance, the inflation detection system can comprise a switch disposed to be moved by the outer wall of the donor reservoir when the donor reservoir is inflated with oxygen to the predetermined state of inflation. The switch can be biased, such as by gravity, by a resiliently compressible member, or by any other effective method, toward the donor reservoir. The switch can be considered to have an activated state wherein the switch is disposed at or beyond an inward position with respect to the inner volume of the donor reservoir and a deactivated state when the switch is moved outwardly by the outer wall of the donor reservoir when the volume of oxygen in the donor reservoir reaches the predetermined state of inflation. The valve system is operative to prevent oxygen from flowing from the source of oxygen and into the donor reservoir when the switch is in the deactivated state, and the valve system is operative to permit oxygen to flow from the source of oxygen and into the donor reservoir when the switch is in the activated state.

In particular manifestations of the system, the switch comprises a float switch. For example, the float switch can have a contact structure with a collar that is extendable and retractable relative to a central column. The collar can then retain a magnet, and the central column can then retain electrical contacts that are brought into electrical contact by a proximity of the magnet when the switch is in the activated state.

According to practices of the system, the valve system can take the form of a solenoid valve that is in electrical communication with the inflation detection system. The solenoid valve can be induced by the inflation detection system to a closed condition to prevent the flow of oxygen from the source of oxygen to the donor reservoir when the donor reservoir is in the first condition, and the solenoid valve can be induced by the inflation detection system to an open condition to permit the flow of oxygen from the source of oxygen to the donor reservoir when the donor reservoir is in the second condition.

In certain embodiments, the donor reservoir is disposed within a housing, which could comprise a main housing of the system, a sub-housing within a main housing, or some other type of housing. In other practices, the donor reservoir can be disposed without a housing. Where a housing is provided, the inflation detection system can comprise an electro-mechanical system with a switch supported by the housing and disposed to be moved by the outer wall of the donor reservoir when the donor reservoir is inflated with oxygen to the predetermined state of inflation. In alternative practices of the invention, the inflation detection system comprises a contactless detection system. For instance, the inflation detection system can take the form of an optical detection system. In certain embodiments, all or part of the housing can be transparent. With that, the state of inflation of the donor reservoir can be visually perceived, which can be of further assurance to the user that the system is in proper operation.

A recipient delivery device, such as the nasal cannula, a patient breathing mask, or another recipient delivery device, is coupled to the second end of the ambient pressure conduit. The nasal cannula is constructed to permit breath to be exhausted directly through the cannula and to prevent exhausted breath from being returned into the connected ambient pressure conduit. As taught herein, the nasal cannula permits direct and immediate control over the ratio of oxygen and entrained air inhaled through the nasal cannula during inspiration.

One will appreciate that the foregoing discussion broadly outlines the more important goals and features of the invention to enable a better understanding of the detailed description that follows and to instill a better appreciation of the inventors' contribution to the art. Before any particular embodiment or aspect thereof is explained in detail, it must be made clear that the following details of construction and illustrations of inventive concepts are mere examples of the many possible manifestations of the invention.

### BRIEF DESCRIPTION OF THE DRAWINGS

In the accompanying drawing figures:

FIG. 1 is a schematic view of an oxygen delivery and conservation system according to the present invention;

FIG. 2 is a forward perspective view of the housing portion of an oxygen delivery and conservation system as taught herein;

FIG. 3 is a rearward perspective view of the housing portion of the oxygen delivery and conservation system;

FIG. 4 is a sectioned view in side elevation of an oxygen delivery and conservation system pursuant to the invention;

FIG. 5 is a rearward perspective view of the oxygen delivery and conservation system with the top, bottom, and sidewalls of the housing removed;

FIG. 6 is a schematic view of the oxygen delivery and conservation system in operation during a series of respiratory cycles;

FIG. 7 is a perspective view of a gas blending apparatus for an ambient-pressure gas delivery and conservation system as disclosed herein;

FIG. 8 is a view in front elevation of the gas blending apparatus of FIG. 7;

FIG. 9 is a top plan view of the gas blending apparatus of FIG. 7;

FIG. 10 is an exploded perspective view of the gas blending apparatus of FIG. 7;

FIG. 11 is an alternative exploded perspective view of the gas blending apparatus of FIG. 7; and

FIG. 12 is an exploded view in side elevation of an alternative gas blending apparatus according to the invention.

### DETAILED DESCRIPTION OF PREFERRED EMBODIMENTS

The gas blending apparatus and the ambient-pressure gas delivery and conservation system with which it is operative are subject to a wide variety of embodiments. However, to ensure that one skilled in the art will be able to understand and, in appropriate cases, practice the inventions disclosed herein, certain preferred embodiments of the broader inventions are described below and shown in the accompanying drawing figures.

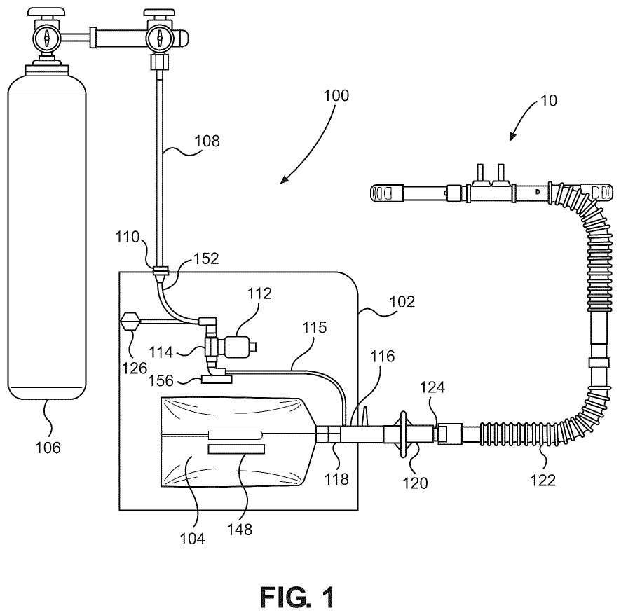

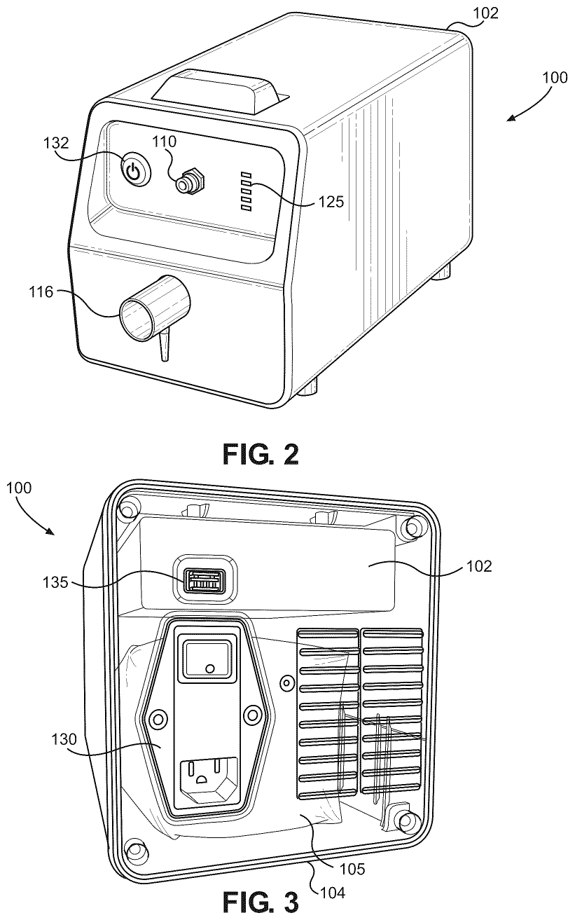

To understand and appreciate the utility and operation of the gas blending apparatus disclosed herein, reference will first be had to the oxygen delivery and conservation system 100 relative to which it is designed to function. One will have reference to the schematic view of FIG. 1 and to the depictions of the delivery dispensing and conservation system 100 of FIGS. 2 through 5 where the gas blending apparatus is indicated generally at 10. There, the illustrated oxygen delivery and conservation system 100, which again may alternatively be referred to as a gas dispensing and conservation system 100, provides an on-demand supply of oxygen at ambient pressure to a recipient. In the depicted example, oxygen is delivered to a recipient through a nasal cannula 158 from a donor reservoir 104 with it being understood that other gas delivery mechanisms, including by way of non-limiting example a breathing mask, would be within the scope of the invention except as it may be expressly limited by the claims.

According to practices of the invention, the donor reservoir 104 retains oxygen at ambient pressure and is continually and automatically supplied with oxygen from an oxygen source 106, such as a tank of compressed oxygen gas or liquid oxygen. With the donor reservoir 104 retaining oxygen at ambient pressure and with the donor reservoir 104 being automatically replenished, a full and ample supply of oxygen is constantly available for patient inspiration. Concomitantly, since oxygen is withdrawn from the reservoir 104 only through inspiration, oxygen losses during patient expiration are substantially eliminated. The supply of oxygen is thus conserved without compromising availability to the individual recipient.

The donor reservoir 104 in this embodiment comprises an expandable and compressible shell, bladder, or other expandable and compressible body that is disposed within a housing 102. The housing 102 could be a primary housing or a sub-housing within a larger structure. However, the donor reservoir 104 need not necessarily be within a housing 102 to be within the scope of the invention. The housing 102 in the depicted embodiment defines an enveloping boundary for the reservoir 104 so that the shell of the reservoir 104 presses toward one or more portions of the boundary defined by the housing 102 as the reservoir 104 is expanded. In the depicted, non-limiting example, the housing 102 has a bottom that defines a lower boundary for the reservoir 104, a top that defines an upper boundary for the reservoir 104, and distal ends that define longitudinal boundaries for the reservoir 104.

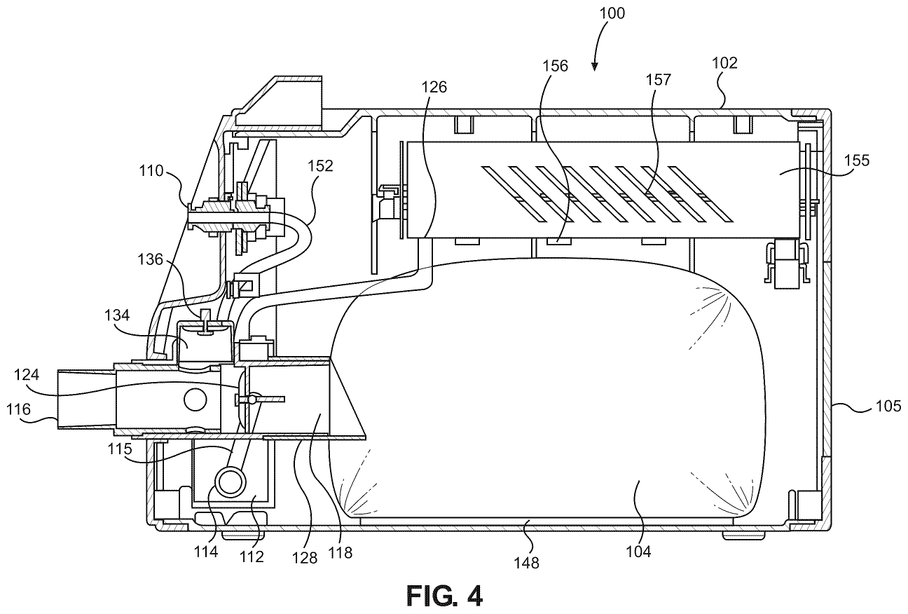

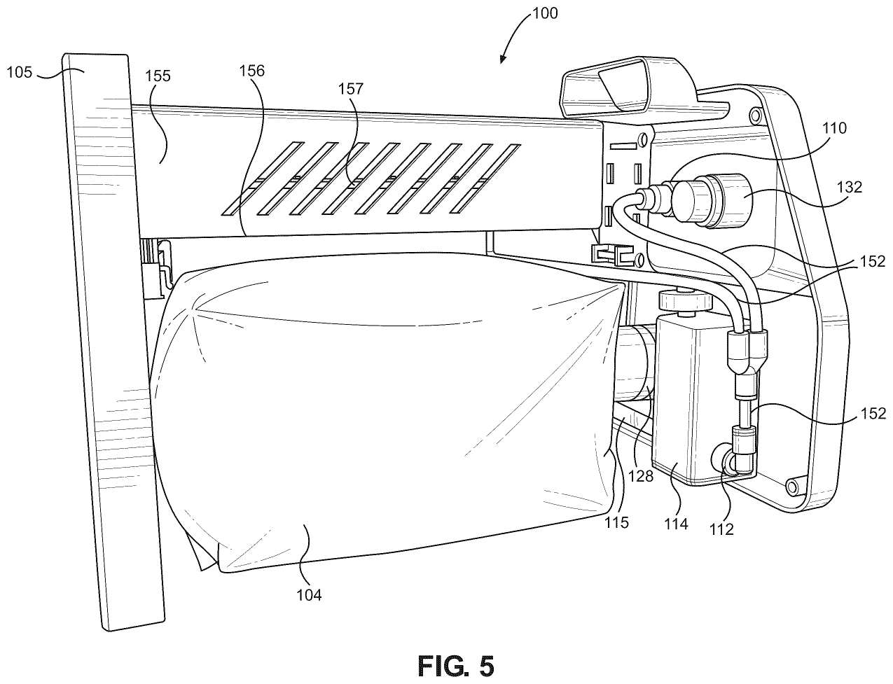

As shown in FIGS. 4 and 5, the housing 102 in this example defines an elongate, generally cubic inner volume, and the reservoir 104 has a corresponding elongate, generally cubic shape. Other shapes and combinations of shapes are readily possible and within the scope of the invention except as it might be expressly limited by the claims. In certain practices of the invention, the lower wall portion of the shell of the reservoir 104 can be adhered or otherwise secured to the housing 102 in one or more locations, such as by an adhesive strip 148 as depicted in FIG. 4 or in any other manner. In this example, the reservoir 104 has four elongate sidewalls joined to exhibit a rectangular cross section when inflated, a first end wall formed by four triangular portions that extend from the first ends of the sidewalls and joined, and a second end wall likewise formed by four triangular portions that extend from the second ends of the sidewalls and joined. The edges of the walls are joined in a sealed manner to define the cubic reservoir 104. The reservoir 104 is thus sealed but for an entry orifice at the first end of the reservoir 104.

The shell of the reservoir 104 is formed from a flexible and substantially gas impermeable material. One or ordinary skill in the art would be aware of numerous such materials. Each is within the scope of the invention except as expressly excluded by the claims. The shell of the reservoir 104 could be formed from a flexible polymeric material with or without a lining layer. The material defining the reservoir 104 could, for example, comprise a foil formed by one or more layers of polymeric material with an aluminum lining. The reservoir 104 can have combinations including one or more flexible walls, rigid walls, compressible walls, collapsible walls, expandable walls, thin walls, or other walls capable of keeping a volume gas inside. Other formations of the reservoir 104 are possible and within the scope of the invention.

Preferably, as is enabled by formation of the reservoir 104 of a lightweight, flexible foil, the reservoir 104 once expanded tends to substantially maintain an expanded shape and configuration, whether by its own structural integrity or otherwise, even when it is open to ambient pressure, such as by a fluidic connection to the recipient, such as through the nasal cannula 158, a breathing mask, or another mechanism for conveying oxygen to a recipient through ambient pressure tubing 122. Since the system 100 is designed to provide oxygen on demand during nature inspiration by the patient, the ambient pressure tubing 122 has a large inner diameter to reduce any resistance of patient inhalation to near zero. As taught herein, once expanded, the reservoir 104 in preferred embodiments does not significantly collapse on its own due to the weight of its walls. When filled with oxygen, the reservoir 104 thus temporarily stores a compartmented volume of oxygen at ambient pressure waiting to be drawn therefrom by the recipient.

In the embodiment of the oxygen dispensing and conservation system 100 of FIG. 1, a fluidic connector 118, which may be considered a T-connector, has a first, longitudinal port in fluidic communication with the donor reservoir 104, such as through a tubular connector 128 that is fixed and sealed within the aperture in the neck of the reservoir 104. The fluidic connector 118 has a second, longitudinal port comprising an output connector 116 in fluidic communication with the ambient pressure tubing 122 and, through that tubing 122, the recipient, such as through the nasal cannula 158, a breathing mask, or another gas delivery mechanism. Finally, the fluidic connector 118 has a third, lateral port between the first and second openings in fluidic communication with the oxygen source 106 in this case through a flow-limiting connector 115. The first, second, and third ports are in fluidic communication with one another within the fluidic connector 118. The fluidic communication from the source 106 to the connector 118 could, for instance, be through high-pressure tubing 108 from the oxygen source 106 to an oxygen connector 110 fixed to the housing 102 and high-pressure tubing 152 from the oxygen connector 110 to a supply valve 112. A pressure sensor 126 is interposed to detect gas pressure entering the supply valve 112, such as by being fluidically interposed along the high-pressure tubing 152.

The supply valve 112, which in this example comprises an electromechanical solenoid valve 112, has an open condition and a closed condition. The valve 112 is fluidically interposed between the pressurized oxygen source 106 and the reservoir 104. When the supply valve 112 is in the open condition, oxygen can be passed from the oxygen source 106, through the tubing 108, through the valve 112, through the connector 118 or gas blending apparatus 10 as disclosed herein, and into the reservoir 104. When the valve 112 is in the closed condition, the passage of oxygen between the oxygen source 106 and the reservoir 104 is prevented.

In the embodiment of FIG. 1, a one-way inspiratory valve 124 is interposed between the reservoir 104 and the recipient, such as by being fluidically connected to the second port of the fluidic connector 118 directly or with a gas filter 120 interposed therebetween. In FIG. 1, the fluidic connector 118 is fluidically connected through its first port to the neck of the reservoir 104. The one-way inspiratory valve 124 is operative to enable gas to flow from the donor reservoir 104, through the ambient pressure tubing 122, and to the recipient, such as through the nasal cannula 158 or a breathing mask, but to prevent reverse gas flow, such as from the recipient and into the donor reservoir 104. The gas filter 120 is fluidically interposed between the recipient and the donor reservoir 104.

The volume of gas, in this example oxygen, in the donor reservoir 104 is retained substantially at ambient pressure. Ambient pressure can be defined as the pressure of the air surrounding the donor reservoir 104. Substantially at ambient pressure may be understood to be equal to or within insubstantially different range of ambient pressure. For instance, substantially at ambient pressure may be interpreted as being within five percent of ambient pressure. As a recipient undergoes the inspiratory phase of breathing, oxygen will be drawn from the donor reservoir 104 through the ambient pressure tubing 122 thereby drawing from and tending to reduce the volume of oxygen in the donor reservoir 104. Due to the compressible nature of the donor reservoir 104, the reservoir 104 will tend to contract. When it does contract, the donor reservoir 104 is automatically replenished with oxygen or, potentially, another gas. In the present embodiment, inflation of the donor reservoir 104 is triggered by an inflation detection system that detects when the donor reservoir 104 is not fully inflated and that actuates the supply valve 112 to an open condition to inflate the donor reservoir 104 while avoiding pressurization of the reservoir 104 so that the oxygen within the reservoir 104 remains substantially at ambient pressure.

The inflation detection system has a first condition wherein replenishing oxygen is not supplied to the donor reservoir 104 and a second condition wherein replenishing oxygen is supplied to the donor reservoir 104. The first condition can be a condition wherein the donor reservoir 104 is inflated with oxygen to a certain, predetermined state of inflation, and the second condition can be a condition wherein the donor reservoir 104 is inflated with oxygen below the predetermined state of inflation. The inflation detection system is operative to detect when the donor reservoir 104 has reached the predetermined state of inflation. The predetermined state of inflation can be detected when the donor reservoir 104 reaches a predetermined size or other inflation condition in any dimension or combination of dimensions. In embodiments of the invention, the donor reservoir 104 can be considered to have a fully inflated condition, and the inflation detection system detects when the donor reservoir 104 is inflated to the fully inflated condition or to within a predetermined range of the fully inflated condition. By way of example and not limitation, the inflation detection system can detect when the donor reservoir 104 is inflated with oxygen at or above a threshold inflation level, which may be equal to or less than the fully inflated condition.

Made aware of the present invention, one skilled in the art may appreciate plural mechanisms that would operate as inflation detection systems to detect when the donor reservoir 104 is inflated to the predetermined state of inflation. Each such mechanism is within the scope of the invention except as it may be expressly limited by the claims. Inflation detection mechanisms could comprise mechanical systems, electrical systems, electromagnetic systems, optical systems, electro-mechanical systems, sound-activated systems, movement sensors, light sensors, and any other type of system effective to detect when the donor reservoir 104 is inflated to a predetermined state of inflation with it again being noted that the predetermined state of inflation may be reached while the oxygen within the donor reservoir 104 is substantially at ambient pressure.

In the embodiments of FIGS. 1 through 5, the inflation detection system takes the form of a contactless detection system 156, which can be an optical detection system 156. The detection system 156 could be carried forth by, for instance, a laser detection system, a camera system, an infrared inflation detection system, or any other effective contactless detection system 156. In certain embodiments, a contactless detection system 156 can be formed with a light emitter, such as a laser or other light emitter, retained to one side of the reservoir 104 and a light receptor disposed to the opposite side of the reservoir 104.

Under such constructions, the inflation condition of the donor reservoir 104 can be sensed in a contactless manner, such as where the donor reservoir 104 is inflated to a condition where the reservoir 104 prevents the communication of light from the light emitter to the light receptor, where the reservoir 104 demonstrates a predetermined reflectance value, or in some other contactless manner. In other embodiments, the detection system 156 comprises one or more proximity sensors that are operative to detect the proximity of the localized, facing surface of the donor reservoir 104. For instance, as in FIGS. 4 and 5, a series of proximity sensors forming the inflation detection system 156 are retained relative to the interior of the housing 102, in this example, relative to an electronics casing 155 that is fixed within the housing 102 above the donor reservoir 104. The electronics casing 155 retains an electronic control system 157 with an electronic circuit board that retains electronic memory retaining system software, one or more computer processors for processing the software, and further electronic circuitry and wiring necessary for operation of the system 100.

The inflation detection system 156 can thus detect the inflation condition of the donor reservoir 104, potentially at multiple locations therealong. The inflation detection system 156 can detect when the donor reservoir 104 is filled to a predetermined state of inflation. The predetermined state of inflation can be sensed, for example, based on the sensed position of the wall of the donor reservoir 104, such as by a detection of the proximity of the wall of the donor reservoir 104 to the proximity sensors of the inflation detection system 156 or the sensed inflation of the reservoir 104 to obstruct optical communication between an emitter and a receptor.

Based on the state of inflation of the reservoir 104 as detected by the inflation detection system 156, the flow switch 114 is operative to actuate the valve 112 between the ON condition where oxygen is permitted to flow from the oxygen source 106 to the donor reservoir 104 and the OFF condition where such flow is prevented. More particularly, when the donor reservoir 104 is detected by the inflation detection system 156 to be below the predetermined state of inflation based on the inward contractive movement of the walls of the donor reservoir 104, the flow switch 114 will trigger the valve 112 to the ON condition to permit oxygen to flow from the oxygen source 106 to fill the donor reservoir 104. When the donor reservoir 104 reaches the predetermined state of inflation based on a detected expansion of the walls of the donor reservoir 104, again as detected by the inflation detection system 156, the flow switch 114 will trigger the valve 112 to the OFF condition to prevent the further flow of oxygen beyond the predetermined state of inflation thereby preventing pressurization of the donor reservoir 104 and preventing expelling of oxygen or other gas from the system 100. The flow switch 114 thus has an activated state, which may be considered to be the ON condition, when the donor reservoir 104 is detected to be below the predetermined state of inflation, and flow switch 114 has a deactivated state, which may be considered to be the OFF condition, when the donor reservoir 104 is detected to have reached the predetermined state of inflation based on a detection by the inflation detection system 156 of the expansion of the donor reservoir 104.

In another non-limiting embodiment, the inflation detection system comprises an electro-mechanical system for detecting when the donor reservoir 104 is filled to the predetermined state of inflation. Such an inflation detection system (not illustrated in the present embodiment) can have a contact structure disposed to contact, to be contacted by, to be moved by, or otherwise to be actuated by the donor reservoir 104 when the reservoir 104 reaches a stage of inflation. Within the scope of the invention, the location and construction of the contact structure could vary. The contact structure can, for instance, be disposed to project from or through the distal end wall of the housing 102 and into the inner volume of the housing 102 so that it projects toward and can engage the distal end of the reservoir 104. In other embodiments, the contact structure can be disposed to project from or through the upper wall of the housing 102 and into the inner volume of the housing 102 to engage a mid-portion of the reservoir 104. The contact structure can, for example, be retained by a support structure fixed to the upper wall or another upper portion of the housing 102.

The contact structure is positioned to be moved by the donor reservoir 104 as the reservoir 104 expands toward an inflated condition. The contact structure can, for instance, be depressed, pivoted, rotated, or otherwise actuated by the donor reservoir 104 and more particularly by an expansion of the donor reservoir 104. The contact structure can then operate as or as a component of or to actuate a flow switch 114. When the contact structure is actuated by the expansion of the donor reservoir 104, the flow switch 114 is operative to actuate the valve 112 between the ON condition where oxygen is permitted to flow from the oxygen source 106 to the reservoir 104 to replenish and fill the reservoir 104 and the OFF condition where oxygen is prevented from flowing from the oxygen source 106 to the reservoir 104. The contact structure can be biased, such as by spring force, under the force of gravity, by resiliency, or any other biasing method or combination thereof toward the donor reservoir 104.

In the non-limiting embodiment of FIGS. 1 through 5, the donor reservoir 104 is disposed within a housing 102. Additionally or alternatively, the donor reservoir 104 could be disposed within a sub-housing that, in turn, could be disposed in the housing 102 or that could stand independently. Still further, the donor reservoir 104 could be disposed without a housing or enclosure, in which case the contact structure and potentially the flow switch could be otherwise retained, such as by a surrounding band, a rigid arm, or another retaining structure, for contact or other sensing or engagement relative to the donor reservoir 104. The contact structure and the flow switch 114 could be retained together, potentially as a unit, or in separate locations.

The contact structure is permitted to move inwardly in the direction toward the donor reservoir 104 when the volume of oxygen in the donor reservoir 104 falls below a predetermined state of inflation such that the outside wall is, or can be, deflected or moved inwardly. The flow switch 114 has an activated state, which may be considered to be the ON condition, when the contact structure is sufficiently moved, such as by extension, pivoting, or other movement, in an inward direction toward the inner volume of the donor reservoir 104. The flow switch 114 has a deactivated state, which may be considered to be the OFF condition, when the contact structure is moved, such as by retraction, pivoting, or other movement in an outward direction away from the donor reservoir 104. The contact structure is moved outwardly to adjust the flow switch 114 to the deactivated state, which is the OFF condition, when the volume of oxygen in the donor reservoir 104 reaches the predetermined state of inflation to cause the outside wall of the donor reservoir 104 to be advanced outwardly by the expansion of the donor reservoir 104. For instance, where the contact structure is a depression switch, expansion of the donor reservoir 104 will press the outer wall or shell of the donor reservoir 104 outwardly to press the contact structure and the flow switch 114 to the deactivated state.

With each inflation detection system, the donor reservoir 104 can thus be inflated, such as to or within a given range of the maximum volume of the donor reservoir 104 without over-inflation or pressurization of the donor reservoir 104. Oxygen within the donor reservoir 104 is thus prevented from exceeding approximately ambient pressure. Except as might otherwise be required by the claims, however, embodiments of the invention could calibrated to induce the flow switch 114 and the valve 112 to the deactivated state at some other predetermined inflation condition or pressure, including potentially a pressure or inflation condition in excess of ambient pressure or to some inflation condition well below the maximum volume of the donor reservoir 104. The flow switch 114 and the valve 112 can be electrical, mechanical, electro-mechanical, or otherwise configured and constructed.

In embodiments of the oxygen dispensing and conservation system 100, the supply valve 112 can comprise a solenoid valve that is in electrical communication, such as through electrical wiring in an electrical circuit, with the flow switch 114. As illustrated, an electrical control system 157, which can include electrical circuitry, electronic memory, wiring, system software retained and operative by electrical circuitry and electronic memory, and other electrical control and connection components, cooperates with the inflation detection system to induce the solenoid supply valve 112 to an open condition to permit the flow of oxygen from the source 106 when the flow switch 114 is in the activated state. The electrical control system can receive power from a power source, which could be a source of alternating current through a power supply connection 130, a source of direct current such as a battery power source, or some other source of electric power. The flow of electrical power from the power source can be controlled by a power switch 132. The solenoid valve 112 is induced by the inflation detection system and the electrical control system to a closed condition to prevent the flow of oxygen from the source 106 to the reservoir 104 when the flow switch 114 is in the deactivated state. Each of the components referenced herein can be further combined or separated within the scope of the invention.

Even when the valve 112 is in an open condition, the rate of flow, the pressure of flow, or both the pressure and rate of flow of oxygen from the source 106 to the donor reservoir 104 are limited by the flow-limiting connector 115. The flow-limiting connector 115 could limit the flow rate of oxygen from the source 106 to the donor reservoir 104 to a predetermined flow rate, such as less than 1 liter per minute or any other flow rate. The flow-limiting connector 115 could, for example, comprise a narrow-diameter tube connector, such as a connector having an inner diameter of 0.02 mm or some other dimension reduced as compared to other conduit connections within the fluidic system. Rapid changes in pressure within the donor reservoir 104 can thus be prevented on opening of the valve 112.

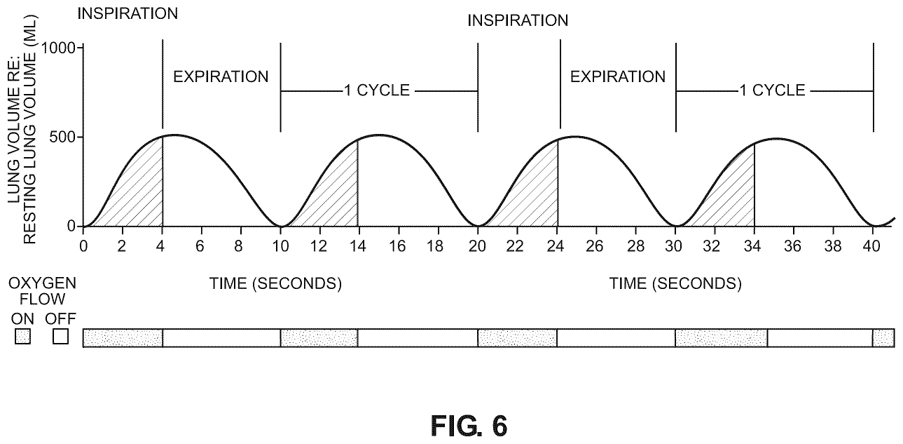

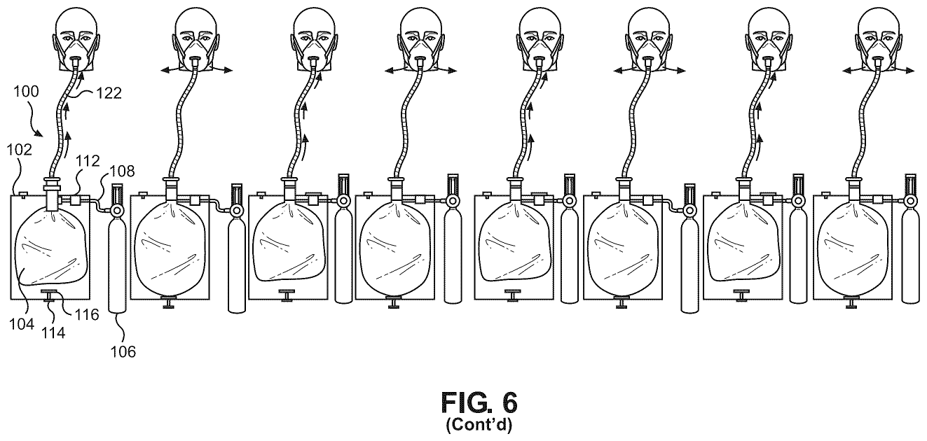

Referring to FIG. 6, the system 100 is depicted in operation during a series of respiratory cycles to provide an on-demand supply to a recipient, such as through a mask or the nasal cannula 158 worn by a patient in need. In operation of the system 100, inspiration by the patient will operate to draw oxygen at ambient pressure from the donor reservoir 104 thereby tending to contract the reservoir 104. When the reservoir 104 falls below the predetermined state of inflation, oxygen is allowed to flow and the reservoir 104 is automatically filled to the predetermined state of inflation. A volume of continually-replenished oxygen at ambient pressure is thus available within the reservoir 104 to be drawn through the ambient pressure tubing 122 during a natural inspiration phase of a breathing cycle. When the recipient is not engaged in inspiration, no oxygen is drawn and no oxygen tends to be expelled from the reservoir 104. When the volume of oxygen within the reservoir 104 falls below the predetermined state of inflation, the inflation detection system will detect the same and trigger the valve 112 to an open condition. Flow of oxygen is then permitted from the oxygen source 106 so that the donor reservoir 104 will be filled with oxygen until the predetermined state of inflation is reached. When the predetermined state of inflation is reached, the inflation detection system will detect the same and trigger the valve 112 to a closed condition to prevent the further supply of oxygen to the donor reservoir 104 from the source 106 until a further inspiration phase of a breathing cycle draws a volume of oxygen from the reservoir 104. The donor reservoir 104 is thus automatically supplied with oxygen while pressurization of the oxygen in the reservoir 104 is automatically prevented. Supplemental oxygen is safely and effectively supplied to the patient at ambient pressure in an on-demand volumetric displacement system enabling the transfer of oxygen during the entire inspiratory phase of the breathing cycle while the wasteful release of oxygen to the atmosphere is prevented, including during the expiratory phase.

The donor reservoir 104 thus automatically receives replenishing oxygen from the pressurized source 106 through the high-pressure tubing 108 and through the supply valve 112. The automatic refilling of the reservoir 104 ensures that the donor reservoir 104 always retains a supply of oxygen available for the next inspiratory phase of the breathing cycle while the oxygen in the reservoir 104 never exceeds ambient pressure. Where the donor reservoir 104 is visually exposed, such as through a partially or completely transparent housing 102 or an observation aperture or window in the housing 102, an observer is provided with visual confirmation of the state of inflation of the donor reservoir 104. For example, as FIG. 3 illustrates, the housing 102 of the depicted embodiment has a translucent rear wall portion 105 that permits one to observe the inflation condition of the donor reservoir 104, which can be of useful comfort in confirming proper operation in view of the quiet operation of the ambient-pressure oxygen dispensing and conservation system 100. Further, the system 100 includes an electronic status indicator 125 as is shown in FIG. 2 to confirm the state of inflation of the donor reservoir 104. The electronic status indicator 125 can, for example, comprise a series of light sources or another visual electronic indicator of the level to which the donor reservoir 104 is filled. The system 100 can thus provide a synchronized delivery of supplemental oxygen to a recipient as the donor reservoir 104 and the system 100 in general synchronize with the physiological ventilations of a patient based on the storage and replenishment of oxygen in the donor reservoir 104 at ambient pressure and the termination of the supply of oxygen automatically when the donor reservoir 104 reaches the predetermined state of inflation.

Within the scope of the invention, the system 100 can measure, record, and analyze the flow of oxygen and the breathing characteristics of a patient, including by use of the electrical control system 157 and, potentially or alternatively, through remote data communication and data processing by wireless communication. As shown in FIG. 3, a data port 135, such as a USB port or another data port, allows wired communication to and from the electrical control system 157 and the system 100 in general. By wired or wireless communication using the data port 135 or a wireless communication protocol, system software can be updated and modified, the system 100 can be programmed and adapted to particular requirements, and acquired data, such as data regarding system operation, user respiration, and other aspects, can be downloaded for use and analysis.

To facilitate such data acquisition and analysis, a volumetric measuring flow meter could be connected to the source 106 of oxygen. Additionally or alternatively, one or more flow meters could be retained within the housing 102 along the path of gaseous flow through the system 100. For instance, a flow meter could be disposed to measure oxygen passing through the valve 112. The valve 112 can incorporate a flow meter, or a flow meter could be otherwise disposed. A flow meter could further or alternatively be disposed between the reservoir 104 and the ambient pressure tubing 122. By measuring the volume of oxygen supplied to a recipient by the system 100, such as over a given time period, per cycle of inspiration and expiration, or otherwise, plural determinations, measurements, and analyses can be made. For instance, one can determine the volume of oxygen inspired by the patient and, additionally or alternatively, the volume of oxygen remaining in the oxygen source 106. Through electronic memory and software operating on the electrical system or in communication therewith, whether by direct incorporation, wireless communication, or a combination thereof, the system 100 can harvest, process, and analyze data based on usage of the system 100.

While a compressed gas tank is often depicted and referred to as the oxygen source 106 herein, other oxygen sources 106 are possible within the scope of the invention. By way of further, non-limiting examples, the system 100 can provide on-demand oxygen to patients with oxygen supplied by an oxygen concentrator, which takes in air and removes the nitrogen from it thereby leaving the oxygen-enriched gas for those patients requiring medical oxygen. The typical flow of this compressed oxygen is 1-5 liters/minute. High-end oxygen concentrators can deliver upwards of 50 L/minute, but they require more electricity and more maintenance.

Where the oxygen source 106 is an oxygen concentrator, the system 100 can be placed between the oxygen concentrator and the oxygen delivery device so that, as oxygen leaves the concentrator, it enters the large reservoir 104 where it remains at ambient pressure until the patient inhales. As the patient breathes in and draws oxygen from the reservoir 104, the reservoir 104 begins to deplete, the supply valve 112 from the oxygen concentrator as the oxygen source 106 opens to replenish the reservoir 104 with compressed oxygen from the oxygen concentrator. As the patient exhales, no flow occurs between the reservoir 104 and the patient. Rather than wasting the oxygen flowing from the concentrator during the exhalation phase of the patient, the flow is employed to replenish the reservoir 104. Once the reservoir 104 is full, the supply valve 112 stops the flow of oxygen from the oxygen source 106. The cycle can repeat with every breath. In this manner, oxygen not taken in by the patient during inspiration is stored rather than lost.

As taught herein, oxygen from the donor reservoir 104 can be made available for inspiration by a recipient through a breathing mask, a nasal cannula 158 as shown in FIG. 1, or another gas delivery device. In the nasal cannula 158, first and second straps extend laterally in opposite directions from a central portion thereof, and buckles are retained at the distal ends of the straps. The nasal cannula 158 can be retained relative to the head of a wearer by the straps and buckles, possibly in combination with an additional fastening mechanism, such as a further strap, cloth tape, or any other fastening method. First and second nasal prongs extend in parallel from the central portion of the nasal cannula 158 for being received into the nares of a patient. The nasal cannula 158 has a gas reception aperture for fluidically engaging the ambient pressure tubing 122 of the oxygen dispensing and conservation system 100.

The ambient-pressure gas dispensing and conservation system 100 so disclosed is operative to provide an ample supply of oxygen or other gas on demand without the waste involved in constant flow systems and without the complex mechanisms inherent in pulse dose systems and the difficulties of such systems in tracking natural variations in breathing frequency and volume due to physical activity and other factors. However, as previously described, there is a further need in oxygen supply systems to entrain air with the supplied oxygen to achieve a desired FiO2 oxygen concentration. The prior art has been limited in its ability to do so in a manner that is immediately responsive and consistent, particularly during variations in, for instance, ventilation rate and inspiratory volume.

In the embodiment of the gas dispensing and conservation system depicted in FIGS. 2 through 5, ambient air can be selectively entrained with oxygen drawn from the donor reservoir 104 at a consistent level without regard to ventilation rate, inspiratory volume, and other factors by operation of the gas blending apparatus indicated generally at 10. There, the gas blending apparatus 10 is operative as a fluidic connector. The gas blending apparatus 10 is operative to receive oxygen supplied through the flow-limiting connector 115 from the source of oxygen, to permit the flow of oxygen into and out of the donor reservoir 104, and to provide an output connection for supplying blended air and oxygen to a recipient through ambient pressure tubing 122.

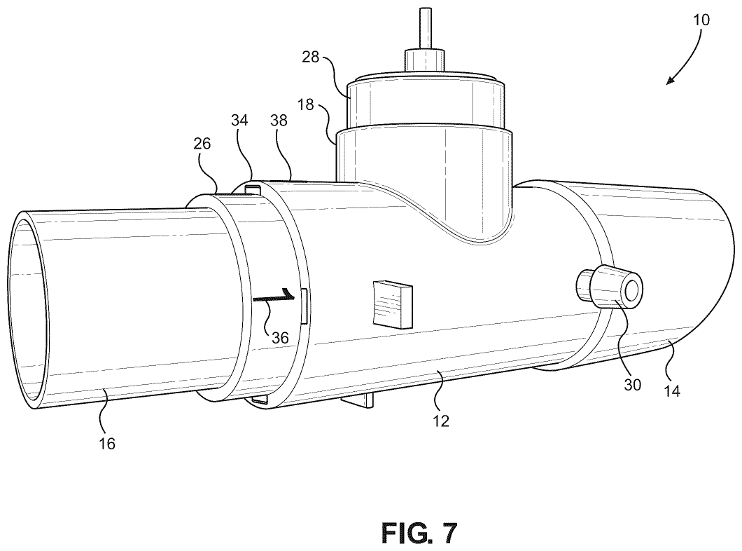

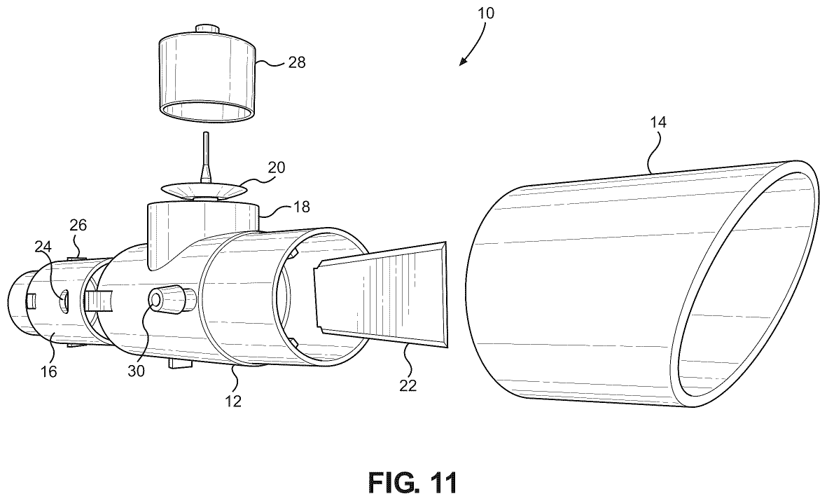

The gas blending apparatus 10 is depicted as incorporated within the gas dispensing and conservation system 100 in FIG. 4, for example, and the gas blending apparatus 10 is depicted apart from the remainder of the dispensing and conservation system 100 in FIGS. 7 through 12. There, the gas blending apparatus 10 can be seen to be founded on a main conduit body 12. In this non-limiting example, the main conduit body 12 is tubular. A first end of the main conduit body 12 establishes a first port that is disposed in fluidic communication with the donor reservoir 104. In the depicted embodiment, the connection between the conduit body 12 and the donor reservoir 104 is established through a tubular neck connector 14 of the gas blending apparatus 10 that is sealingly received in the neck aperture of the donor reservoir 104. The neck connector 14 is matingly engaged with the first end of the conduit body 12. The gas blending apparatus 10 has an output connector 16 disposed at a second end of the conduit body 12. The output connector 16 establishes a second port for supplying gas to a recipient, such as through ambient-pressure tubing 122 as FIG. 1 illustrates.

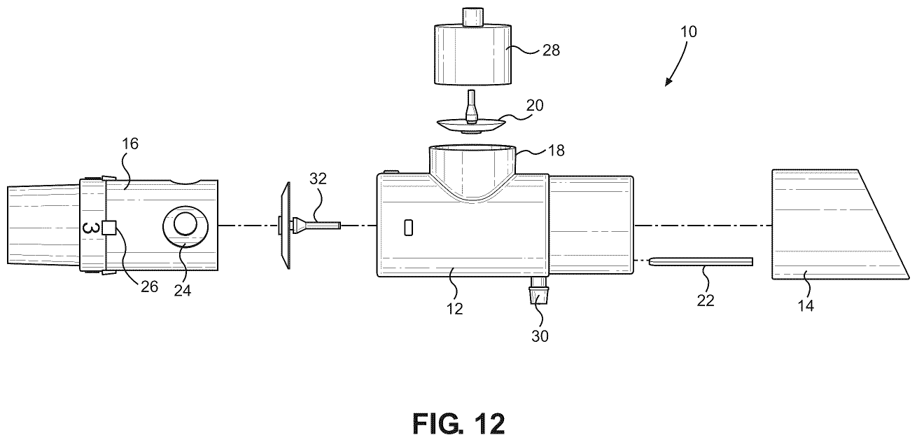

As best seen in FIG. 12, a one-way inspiratory valve 32 is disposed within the main conduit body 12 between the first port of the conduit body 12 in communication with the reservoir 104 and the second port of the conduit body 12 in communication with the output connector 16. The one-way inspiratory valve 32 is operative to allow gas to be drawn from the reservoir 104 through the first port of the conduit body 12 and to be provided to a recipient through the second port of the conduit body 12 while preventing gas from being passed from the second port of the conduit body 12 and into the reservoir 104 through the first port of the conduit body 12, such as through the ambient-pressure tubing 122 during exhalation.

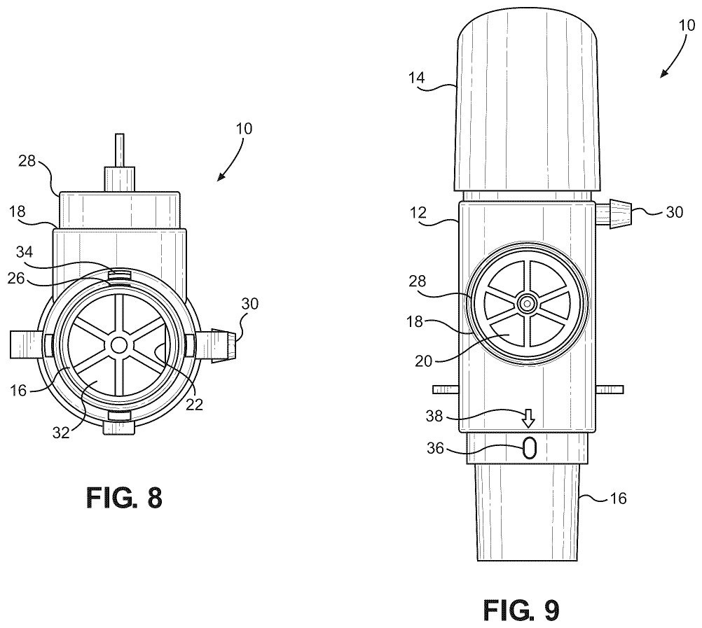

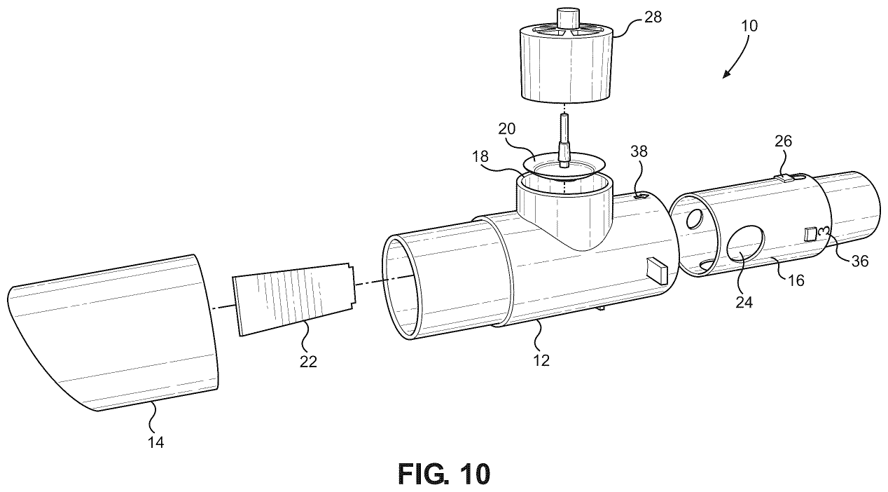

Oxygen or potentially another gas or combination of gases is injected into the gas blending apparatus 10 from a source thereof through an injection port 30. As in FIG. 4, for example, oxygen can be supplied from the oxygen source to the injection port 30 through a supply conduit 115, such as a flow-limiting connector 115. The injection port 30 is disposed proximal to the one-way inspiratory valve 32 with respect to the donor reservoir 104 thereby to direct gas injected through the injection port 30 to act as a replenishing supply for the donor reservoir 104 rather than immediately being discharged through the second port of the conduit body 12 and the output connector 16 and thus to the recipient. As FIGS. 8 and 10 show, a partition panel 22 is longitudinally received into the main conduit body 12 to be disposed with a surface adjacent to the injection port 30 further to direct gas injected through the injection port into the donor reservoir 104.

An air-input orifice 18 is disposed distal to the one-way inspiratory valve 32 with respect to the donor reservoir 104 and thus proximal to the output connector 16 with respect to the one-way inspiratory valve 32 thereby permitting the entrance of ambient air into the gas blending apparatus 10 while preventing that air from entering the donor reservoir 104. A one-way air input valve 20 is fitted to the air-input orifice 18 with a vented cap 28 retained thereover. The donor reservoir 104 can thus be replenished with oxygen or potentially another gas or gases through the injection port 30 and the supply conduit 115. Oxygen or other gas retained within the donor reservoir 104 can be drawn therefrom during inspiration through the output connector 16 while ambient air is selectively entrained therewith through the air-input orifice 18. During expiration, expired breath is prevented from entering the donor reservoir 104 by the one-way inspiratory valve 32.

Thus, when the air-input orifice 18 is open, air will be drawn through the air-input orifice 18 to be blended and entrained with gas drawn from the donor reservoir 104 by operation of the gas blending apparatus 10. The ratio of ambient air drawn through the air-input orifice 18 and gas drawn from the reservoir 104 will be dependent on factors including the effective size of the aperture through the air-input orifice 18 and the effective size of that aperture in relation to the aperture bounded by the neck connector 14 in the donor reservoir 104.

According to the present invention, the effective size of the aperture provided by the air-input orifice 18 is selectively adjustable thereby to permit an adjustment of the ratio of ambient air drawn through the air-input orifice 18 and blended with gas drawn from the reservoir 104. Made aware of the present disclosure, one of ordinary skill in the art would be aware of plural mechanisms for selectively adjusting the effective size of the aperture provided by the air-input orifice 18. Each is within the scope of the invention except as may be expressly excluded by the claims.

In the embodiment of the gas blending apparatus 10 as depicted, for example, in FIGS. 4 and 7 through 12, adjustment of the effective aperture provided by the air-input orifice 18 is provided by a rotary adjustment dial formed by the output connector 16 in combination with the main conduit body 12. More particularly, the output connector 16 has a cylindrical proximal portion that is matingly received into the main conduit body 12 to overlap with the air-input orifice 18, and the output connector 16 has a cylindrical distal portion for being matingly engaged with the ambient-pressure tubing 122. The cylindrical proximal portion of the output connector 16 has a plurality of differently sized apertures 24 circumferentially spaced therearound. While different permutations are certainly possible, the apertures 24 in the proximal portion of the output connector 16 in the depicted example sequentially vary in size from an aperture 24 of a maximum size to an aperture 24 of a minimum size.

The apertures 24 are disposed within the output connector 16 to align longitudinally with the air-input orifice 18. Accordingly, by a selective rotation of the output connector 16 in relation to the main conduit body 12, a selected aperture 24 can be circumferentially aligned with the air-input orifice 18. Thus, when the largest aperture 24 of the output connector 16 is selectively aligned with the air-input orifice 18, a maximum or greatest proportion of air will be drawn in through the orifice 18 to blend with the gas drawn from the donor reservoir 104. When the smallest aperture 24 is selectively aligned with the air-input orifice 18, the proportion of air drawn through the orifice 18 to blend with the gas drawn from the donor reservoir 104 will be at its smallest, and intermediate apertures 24 permit proportions of air drawn through the orifice 18 between the smallest and greatest proportions. The output connector 16 also has a circumferential location for alignment with the air-input orifice 18 that is without an aperture 24 thereby to prevent air from being drawn in through the orifice 18 and blended with the gas drawn from the donor reservoir 104. At that setting, no air will be blended with the gas drawing from the donor reservoir 104. Under this construction, the output connector 16 can further be referred to as an orifice adjustment member.

The gas blending apparatus 10 has plural FiO2-setting, positive mechanical engagement formations 26 and 34 retained by the output connector 16 and the main conduit body 12 to permit the output connector 16 acting as an orifice adjustment member to be disposed and retained in a known rotational orientation relative to the main conduit body 12. In the present embodiment, the output connector 16 has a plurality of engagement formations 26 comprising rectangular protuberances that project radially outward from its surface that are circumferentially spaced around the output connector 16, and the main conduit body 12 has a plurality of receiving notches forming the engagement formations 34 circumferentially spaced around the inner surface of the second end of the main conduit body 12. The spacing of the protuberating engagement formations 26 of the output connector 16 matches the spacing of the receiving engagement formations 34. With this, the output connector 16 can be selectively positioned rotationally relative to the main conduit body 12 to provide direct and immediate control over the size of the aperture 24 of the output connector 16 that is aligned with the orifice 18 of the main conduit body 12 and thus to provide directly and immediate control over the effective size of the air-input aperture provided through the orifice 18 for permitting air to be drawn into and blended with the stream of gas provided to the recipient. Selective positioning of the output connector 16 relative to the main conduit body 12 thus provides direct control over the fraction of inspired oxygen (FiO2) or other gas or gases delivered to the recipient.

Still further, as perhaps best seen in FIG. 9, visual FiO2 setting indicators 36 and 38 are disposed on the output connector 16 and the main conduit body 12 to provide a visual indication of the aperture setting of the output connector 16 in relation to the main conduit body 12. In the depicted, non-limiting example of FIG. 9, the main conduit body 12 has a setting indicator 38 comprising an arrow marking while the output connector 16 has plural setting indicators 36 comprising numerical indications, such as 0, 1, 2, and 3 circumferentially spaced therearound to indicate the relative size of the aperture 24 or the lack of an aperture 24 aligned with the air-input orifice 18 when that FiO2 setting indicator 36 of the output connector 16 is aligned with the setting indicator 38 of the main conduit body 12. The effective size of the opening provided through the air-input orifice 18 can thus be adjusted in a known manner to provide immediate control over the mixture of atmospheric air with the inhaled oxygen.

By use of the oxygen dispensing and conservation system 100 and the gas blending apparatus 10, a patient can draw supplemental oxygen from the donor reservoir 104 through a breathing mask, through a nasal cannula 158 as disclosed herein, or through another delivery apparatus. Alternative recipient delivery apparatuses could, for example, comprise laryngeal mask airways (LMA), endotracheal tubes, tracheostomys, ventilator attachments, CPAP machine connectors, Ambu bags, or even delivery devices for oxygen delivered during recreation.

The on-demand supply of oxygen to be naturally inspired that is provided by the donor reservoir 104 with the present system 100 and gas blending apparatus 10 overcomes numerous deficiencies and limitations exhibited by systems of the prior art. For instance, to achieve the prescribed inspired oxygen concentration, many prior art systems are dependent on the patient's peak inspiratory flow rate (PIFR). For example, when a patient requires a low-inspired oxygen concentration, using a nasal cannula at a low continuous flow rate may help, but this practice limits the patient's oxygen to a low inspired oxygen concentration only. Should the patient's oxygen requirements increase significantly, the inspiratory effort to drive more air into the lungs, which is dependent on tidal volume, ‘speed’ of inspiration, and respiratory rate, will make the PIFR exceed the flow rate at which oxygen or an oxygen/air mixture is supplied by the delivery device. This will mean that at the time of PIFR more or less entrainment of room air occurs, altering the resulting FiO2 in an unpredictable fashion. On the other hand, while using a non-rebreathing face mask at very high flows of oxygen (10-15 L/Min) is capable of providing a reliable delivery of oxygen volume at the prescribe concentration while being less dependent on PIFR, large volumes of oxygen are wasted to the environment with oxygen continuing to flow even during expiration.

In a marked development, the oxygen dispensing and conservation system 100 disclosed herein is capable of passively permitting the transfer of oxygen or another gas or gases from the ambient pressure reservoir 104 by making the gas or gases available to the recipient in a manner that matches the exact volume and rate of demand by the recipient. The drop in pressure induced by inhalation is used for the transfer of volume from the reservoir 104. No extra pressure is required, such as to open a pressure check valve, to start the flow as may be required where a chamber or reservoir contains oxygen at a higher pressure than ambient pressure.

Moreover, where the gas blending apparatus 10 is employed, direct and immediate control is provided over the ratio of oxygen and entrained air provided during inspiration. The ambient-pressure oxygen dispensing and conservation system 100 using such a gas blending apparatus 10 enables the achievement of a “shadow effect” wherein oxygen or another gas is provided at a desired saturation on demand with every breath without regard to respiratory frequency, volume, or other factors. The system 100 adjusts to a patient's breathing pattern immediately and automatically. By setting the effective size of the opening provided for drawing air into the gas blending apparatus 10, a consistent fraction of inspired oxygen (FiO2) or other gas or gases is provided to the recipient without a need for complex mechanical or software systems. Consequently, a recipient is able to maintain a desired oxygen-blood saturation (SaO2) even during changes in respiratory frequency and volume. Since the dispensing and conservation system 100 is passive with respect to the ventilation received by the patient, the patient's own breathing effectively controls the amount of oxygen provided at all times. Without requiring complex mechanisms or software and without repeated adjustments, the patient will automatically breathe in approximately the same fraction of inspired gases without regard to the volume or frequency of inspiration.

As used herein, references to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, for example, the term “or” should generally be understood to mean “and/or.” Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately,” and the like, when accompanying a numerical value, are to be construed as indicating a deviation as would be appreciated by one of ordinary skill in the art to operate satisfactorily for an intended purpose. Similarly, words of approximation such as “approximately” or “substantially” when used in reference to physical characteristics should be understood to contemplate a range of deviations that would be appreciated by one of ordinary skill in the art to operate satisfactorily for a corresponding use, function, or purpose. The use of any and all examples or exemplary language, as in “such as” or the like, provided herein, is intended merely to better illuminate the embodiments and does not pose a limitation on the scope of the embodiments. No language in the specification should be construed as indicating any unclaimed element as essential to the practice of the embodiments. In the description, it is understood that terms such as “first,” “second,” “top,” “bottom,” “upper,” “lower,” and the like are words of convenience and are not to be construed as limiting terms.

With certain details and embodiments of the present inventions for a gas blending apparatus 10 and the ambient-pressure oxygen delivery and conservation system 100 with which it is operative disclosed, it will be appreciated by one skilled in the art that numerous changes and additions could be made thereto without deviating from the spirit or scope of the invention. This is particularly true when one bears in mind that the presently preferred embodiments merely exemplify the broader invention revealed herein. Accordingly, it will be clear that those with major features of the invention in mind could craft embodiments that incorporate those major features while not incorporating all of the features included in the preferred embodiments.

Therefore, the following claims shall define the scope of protection to be afforded to the invention. Those claims shall be deemed to include equivalent constructions insofar as they do not depart from the spirit and scope of the invention. It must be further noted that a plurality of the following claims may express, or be interpreted to express, certain elements as means for performing a specific function, at times without the recital of structure or material. As the law demands, any such claims shall be construed to cover not only the corresponding structure and material expressly described in this specification but also all legally-cognizable equivalents thereof.

We claim as deserving the protection of Letters Patent:

1. A gas blending apparatus for use with an ambient pressure gas delivery and conservation system with ambient pressure tubing for providing gas at ambient pressure from a donor reservoir to a recipient, the gas blending apparatus comprising:
a main conduit body with an inner volume, a first port for being disposed in fluidic communication with the donor reservoir, and a second port for supplying gas to the recipient;
a one-way inspiratory valve disposed within the main conduit body between the first port and the second port wherein the one-way inspiratory valve is operative to permit gas to be drawn in from the donor reservoir through the first port and passed through the second port to the recipient and wherein the one-way inspiratory valve is operative to prevent gas from being received through the second port and passed through the first port into the donor reservoir;
an air-input orifice in the main conduit body distal to the one-way inspiratory valve with respect to the first port, wherein the air-input orifice is operative to provide an aperture into the inner volume of the main conduit body with an effective size to permit the entrance of ambient air into inner volume of the gas blending apparatus;
whereby ambient air can be drawn into the main conduit body through the air-input orifice and blended with gas drawn from the donor reservoir in a ratio of ambient air to gas drawn from the donor reservoir.

2. The gas blending apparatus of claim 1, further comprising a neck connector engaged with the main conduit body for coupling the first port of the main conduit body with the donor reservoir.

3. The gas blending apparatus of claim 1, further comprising a one-way valve fitted to the air-input orifice wherein the one-way valve fitted to the air-input orifice is operative to permit ambient air to be drawn into the inner volume of the main conduit body but to prevent gas from being exhausted through the air-input orifice.

4. The gas blending apparatus of claim 1, wherein the effective size of the aperture provided by the air-input orifice is selectively adjustable thereby to permit an adjustment of the ratio of ambient air drawn through the air-input orifice to gas drawn from the donor reservoir through the first port.

5. The gas blending apparatus of claim 4, wherein the effective size of the aperture provided by the air-input orifice is selectively adjustable manually.

6. The gas blending apparatus of claim 5, further comprising an orifice adjustment member selectively engaged with the main conduit body to overlap with the air-input orifice wherein the orifice adjustment member is repositionable in relation to the main conduit body to adjust the effective size of the aperture into the inner volume of the main conduit body provided by the air-input orifice to provide direct and immediate control over the ratio of ambient air to gas drawn through the first port from the donor reservoir.

7. The gas blending apparatus of claim 6, wherein the orifice adjustment member has a plurality of apertures in spaced relation to one another for being selectively aligned with the air-input orifice to adjust the effective size of the aperture provided by the air-input orifice and to provide direct and immediate control over the ratio of ambient air to gas drawn through the first port from the donor reservoir.

8. The gas blending apparatus of claim 7, wherein the orifice adjustment member has a plurality of aperture locations in spaced relation to one another that establish different effective opening sizes for being selectively aligned with the air-input orifice to adjust the effective size of the aperture provided by the air-input orifice and to provide direct and immediate control over the ratio of ambient air to gas drawn through the first port from the donor reservoir.

9. The gas blending apparatus of claim 6, wherein the orifice adjustment member comprises a cylindrical member that is selectively repositionable in relation to the main conduit body to overlap with the air-input orifice.

10. The gas blending apparatus of claim 9, wherein the cylindrical member has a plurality of differently sized apertures in spaced relation for being selectively aligned with the air-input orifice to adjust the effective size of the aperture provided by the air-input orifice and to provide direct and immediate control over the ratio of air drawn through the air-input orifice in relation to gas drawn from the donor reservoir.

11. The gas blending apparatus of claim 10, wherein the differently sized apertures in the cylindrical member are circumferentially spaced around the cylindrical member.

12. The gas blending apparatus of claim 11, wherein the differently sized apertures spaced around the cylindrical member vary in size sequentially.

13. The gas blending apparatus of claim 10, wherein the cylindrical member comprises a portion of an output connector matingly engaged with the main conduit body and wherein the output connector further comprises a tubular portion that extends beyond the main conduit body for connecting to the ambient pressure tubing for providing gas to the recipient.

14. The gas blending apparatus of claim 13, further comprising mechanical engagement formations retained on at least one of the cylindrical member and the main conduit body to permit the cylindrical member to be disposed and retained in a known position relative to the main conduit body.

15. The gas blending apparatus of claim 14, further comprising visual setting indicators retained to move with the cylindrical member and at least one visual setting indicator retained on the main conduit body for permitting the effective size of the aperture provided by the air-input orifice to be adjusted in a known manner.

16. The gas blending apparatus of claim 1, further comprising an injection port in the main conduit body for receiving gas from a supply source wherein the injection port is disposed proximal to the one-way inspiratory valve with respect to the first port.

17. A gas delivery system for providing gas to an individual, the gas delivery system comprising:
a donor reservoir adapted to retain gas wherein the donor reservoir has a fully inflated condition;
a supply valve in fluidic association with the donor reservoir, the supply valve with an open condition wherein gas is allowed to flow into the donor reservoir and a closed condition wherein gas is not allowed to flow into the donor reservoir;
an inflation detection system operative to detect when the donor reservoir is inflated to within a predetermined range of the fully inflated condition, wherein the inflation detection system detects a first condition when the donor reservoir is inflated to within the predetermined range of the fully inflated condition, wherein the inflation detection system detects a second condition when the donor reservoir is inflated below the predetermined range of the fully inflated condition, and wherein the inflation detection system is operative to actuate the supply valve to the open condition when the donor reservoir is inflated below the predetermined range of the fully inflated condition;
a gas blending apparatus comprising: a main conduit body with an inner volume, a first port for being disposed in fluidic communication with the donor reservoir, and a second port for supplying gas to the recipient; a one-way inspiratory valve disposed within the main conduit body between the first port and the second port wherein the one-way inspiratory valve is operative to permit gas to be drawn in from the donor reservoir through the first port and passed through the second port to the recipient and wherein the one-way inspiratory valve is operative to prevent gas from received through the second port from passing through the first port into the donor reservoir; an air-input orifice in the main conduit body distal to the one-way inspiratory valve with respect to the first port, wherein the air-input orifice is operative to provide an aperture into the inner volume of the main conduit body with an effective size to permit the entrance of ambient air into inner volume of the gas blending apparatus;
whereby ambient air can be drawn into the main conduit body through the air-input orifice and blended with gas drawn from the donor reservoir in a ratio of ambient air to gas drawn from the donor reservoir.

18. The gas delivery system of claim 17, wherein the gas delivery system comprises an ambient pressure gas dispensing system, wherein the donor reservoir is adapted to retain gas substantially at ambient pressure, and wherein the inflation detection system is operative to actuate the supply valve to inflate the donor reservoir without pressurization.

19. The gas delivery system of claim 17, wherein the gas blending apparatus further comprises a one-way valve fitted to the air-input orifice wherein the one-way valve fitted to the air-input orifice is operative to permit ambient air to be drawn into the inner volume of the main conduit body but to prevent gas from being exhausted through the air-input orifice.

20. The gas delivery system of claim 17, wherein the effective size of the aperture provided by the air-input orifice of the gas blending apparatus is selectively adjustable thereby to permit an adjustment of the ratio of ambient air drawn through the air-input orifice to gas drawn from the donor reservoir through the first port.

21. The gas delivery system of claim 20, further comprising an orifice adjustment member selectively engaged with the main conduit body to overlap with the air-input orifice wherein the orifice adjustment member is repositionable in relation to the main conduit body to adjust the effective size of the aperture into the inner volume of the main conduit body provided by the air-input orifice to provide direct and immediate control over the ratio of ambient air to gas drawn through the first port from the donor reservoir.

22. The gas delivery system of claim 21, wherein the orifice adjustment member has a plurality of apertures in spaced relation to one another for being selectively aligned with the air-input orifice to adjust the effective size of the aperture provided by the air-input orifice and to provide direct and immediate control over the ratio of ambient air to gas drawn through the first port from the donor reservoir.

23. The gas delivery system of claim 22, wherein the orifice adjustment member has a plurality of aperture locations in spaced relation to one another that establish different effective opening sizes for being selectively aligned with the air-input orifice to adjust the effective size of the aperture provided by the air-input orifice and to provide direct and immediate control over the ratio of ambient air to gas drawn through the first port from the donor reservoir.

24. The gas delivery system of claim 21, wherein the orifice adjustment member comprises a cylindrical member that is selectively repositionable in relation to the main conduit body to overlap with the air-input orifice.

25. The gas delivery system of claim 24, wherein the cylindrical member has a plurality of differently sized apertures in spaced relation for being selectively aligned with the air-input orifice to adjust the effective size of the aperture provided by the air-input orifice and to provide direct and immediate control over the ratio of air drawn through the air-input orifice in relation to gas drawn from the donor reservoir.

26. The gas delivery system of claim 24, wherein the cylindrical member comprises a portion of an output connector matingly engaged with the main conduit body and wherein the output connector further comprises a tubular portion that extends beyond the main conduit body for connecting to an ambient pressure conduit for providing gas to the recipient.

27. The gas delivery system of claim 26, further comprising mechanical engagement formations retained on at least one of the cylindrical member and the main conduit body to permit the cylindrical member to be disposed and retained in a known position relative to the main conduit body.

28. The gas delivery system of claim 17, further comprising an injection port in the main conduit body of the gas blending apparatus for receiving gas from a supply source wherein the injection port is disposed proximal to the one-way inspiratory valve with respect to the first port.
